(19) United States
(12) Patent Application Publication
(10) Pub. No.: US 2016/0244804 A1
(43) Pub. Date: Aug. 25, 2016

(54) METHOD FOR ASSAYING CELLULASE ACTIVITY, SCREENING METHOD USING ASSAYING METHOD, AND HIGH-PERFORMANCE CELLULASE-PRODUCING BACTERIA SELECTED USING SCREENING METHOD

(71) Applicant: SHOWA DENKO K.K., Tokyo (JP)

(72) Inventors: Ichiro FUJITA, Kawasaki-shi (JP); Shinji YAMAKI, Kawasaki-shi (JP); Tadashi YONEDA, Chiba-shi (JP)

(73) Assignee: SHOWA DENKO K.K., Tokyo (JP)

(21) Appl. No.: 15/044,691

(22) Filed: Feb. 16, 2016

(30) Foreign Application Priority Data
Feb. 24, 2015 (JP) 2015-033730

(51) Int. Cl. C12Q 1/34; C12N 1/14; C12R 1/645

(52) U.S. Cl. CPC C12Q 1/34; C12R 1/645; C12N 1/14; G01N 2333/942

(57) ABSTRACT

A method for assaying cellulase activity, including: a process (A) of preparing two or more substrate solutions, which have an identical absorbance measured at an identical wavelength and in which cellulose is dispersed at an identical concentration, and measuring the absorbance of each of the substrate solutions; a process (B) of respectively adding different kinds of enzyme solutions to the substrate solutions, and performing an enzyme reaction under the same conditions; a process (C) of measuring the absorbance of each of the substrate solutions after the enzyme reaction; a process (D) of calculating the absorbance decrease values of the substrate solutions before and after the enzyme reaction; and a process (E) of assaying cellulase activities of the enzyme solutions based on the absorbance decrease values. In the assay of the process (E), it is determined that the enzyme solutions have higher cellulase activity as the absorbance decrease values become larger.

### BACKGROUND OF THE INVENTION

1. Field of the Invention

The present invention relates to a method for assaying cellulase activity, a screening method using the assaying method, and high-performance cellulase-producing bacteria selected using the screening method.

Priority is claimed on Japanese Patent Application No. 2015-033730, filed on Feb. 24, 2015, the content of which is incorporated herein by reference.

2. Description of Related Art

In recent years, bioethanol has been attracting attention as an alternative to fossil resources. Use of non-edible cellulose-containing biomass as a raw material of bioethanol has been examined in addition to edible starch. A main component of the cellulose-containing biomass is cellulose, which is a highly crystalline polymer which forms a hydrogen bond within a molecule and between molecules. For this reason, the fact that it is more difficult to saccharify cellulose-containing biomass than starch is a problem.

Examples of means for improving saccharification performance include production of cellulase having high saccharification performance using microorganisms which produce large amounts of cellulase. Here, a method for simply measuring cellulase activity in order to select cellulase having high saccharification performance is needed.

As the method for simply measuring cellulase activity, a method for measuring cellulase activity through turbidimetry using microfibril cellulose for a substrate for activity measurement is known (for example, refer to Japanese Unexamined Patent Application, First Publication No. S58-202000).

As a method of screening high-performance cellulase-producing bacteria, a method has been reported for measuring the diameter of a dissolution hole formed on a medium after growing cellulase-producing bacteria in a culture base material for assay which is prepared by plating a culture solution in cellulose gel prepared such that cellulose dispersed in water is treated using thiocyanate for desalination (for example, refer to PCT International Publication No. WO2012/057064).

As a method for screening enzyme protein for biomass decomposition focused on a combination of component enzymes of cellulase, a method is known for evaluating a synergistic effect of a combination of enzymes by detecting the size of a hollow (a region which is dyed or decolored due to decomposition of biomass in a solid body) which is formed by reacting component enzymes or proteins, which are to be tested and which are obtained by combining the component enzymes, with the solid body containing the biomass, as biomass decomposition activity (for example, refer to Japanese Unexamined Patent Application, First Publication No. 2009-72102).

### SUMMARY OF THE INVENTION

In the methods disclosed in Japanese Unexamined Patent Application, First Publication No. S58-202000 and PCT International Publication No. WO2012/057064, it is impossible to evaluate the saccharification performance for each enzyme in consideration of a composition ratio of component enzymes while it is possible to evaluate the amount of cellulase activity.

In the method disclosed in Japanese Unexamined Patent Application, First Publication No. 2009-72102, it is assumed that mutant proteins of each component enzyme obtained from a non-cell protein synthesis system are evaluated, but it is impossible to evaluate a culture solution of mutation-producing bacteria in which each component enzyme ratio is unknown.

A more simple and accurate method for assaying cellulase activity is expected in order to perform efficient and prompt screening of microorganisms (hereinafter, abbreviated as “high-performance cellulase-producing bacteria”) which can produce cellulase having high saccharification performance.

An object of the present invention is to solve the problems of the above-described related art. Specifically, the object of the present invention is to provide a simple and accurate method for assaying cellulase activity; a method for screening enzymes and microorganisms which have cellulase activity, using the assaying method, and high-performance cellulase-producing bacteria which are selected using the screening method.

The inventors have repeated intensive studies in order to solve the above-described problems, and as a result, the problems have been solved.

That is, in the present invention, the above-described problems are solved by providing a method for assaying cellulase activity described below; a method for screening enzymes and microorganisms which have cellulase activity, using the assaying method described below; and high-performance cellulase-producing bacteria which are selected using the screening method described below.
[1]A method for assaying cellulase activity, including:
   a process (A) of preparing two or more substrate solutions, which have an identical absorbance measured at an identical wavelength and in which cellulose is dispersed at an identical concentration, and measuring the absorbance of each of the substrate solutions;
   a process (B) of respectively adding different kinds of enzyme solutions to the substrate solutions, and performing an enzyme reaction under the same conditions;
   a process (C) of measuring the absorbance of each of the substrate solutions after the enzyme reaction;
   a process (D) of calculating the absorbance decrease values of the substrate solutions before and after the enzyme reaction; and
   a process (E) of assaying cellulase activities of the enzyme solutions based on the absorbance decrease values.
   in which, in the assay of the process (E), it is determined that the enzyme solutions have higher cellulase activity as the absorbance decrease values become larger.
[2]A method for assaying cellulase activity, includes:
   a process (J) of preparing six or more diluents, which have different dilution ratios for each identical enzyme solution with respect to different kinds of enzyme solutions to be assayed;
   a process (A′) of preparing six or more substrate solutions, which have an identical absorbance measured at an identical wavelength and in which cellulose is dispersed at an identical concentration, and measuring the absorbance of each of the substrate solutions;
   a process (B′) of respectively adding the diluents to the substrate solutions and performing an enzyme reaction under the same conditions;
   a process (C′) of measuring the absorbance of each of the substrate solutions after the enzyme reaction;
   a process (D′) of calculating the absorbance decrease values of the substrate solutions before and after the enzyme reaction; and
   a process (E′) of assaying cellulase activities of the enzyme solutions, in which the process (E′) includes
   a process (K) of creating correlation curves between the dilution ratios of the enzyme solutions and the absorbance decrease values,
   a process (L) of extracting a range in which the dilution ratios and the absorbance decrease values are in a proportional relationship in the correlation curves, and
   a process (F) of assaying the cellulase activity of the enzyme solutions based on the correlation curves in the range and determining that an enzyme solution has higher cellulase activity as the maximum value in the range of the absorbance decrease values becomes larger.
[3]A method for assaying cellulase activity, includes:
   a process (J′) of preparing diluents, which are at dilution ratios of 80% and 100% for each identical enzyme solution with respect to different kinds of enzyme solutions to be assayed;
   a process (A″) of preparing two substrate solutions, which have an identical absorbance measured at an identical wavelength and in which cellulose is dispersed at an identical concentration, and measuring the absorbance of each of the substrate solutions;
   a process (B′) of respectively adding the two diluents to the substrate solutions and performing an enzyme reaction under the same conditions;
   a process (C′) of measuring the absorbance of each of the substrate solutions after the enzyme reaction;
   a process (D′) of calculating the absorbance decrease values of the substrate solutions before and after the enzyme reaction; and
   a process (E″) of assaying cellulase activities of the enzyme solutions, in which the process (E″) includes
   a process (G) of determining that an enzyme solution has higher cellulase activity as the change rate of the absorbance decrease values at dilution ratios of 80% to 100% of the enzyme solutions becomes larger.
[4] The method for assaying cellulase activity according to [3], may further include:
   the process (F) according to [2],
   in which it may be determined that an enzyme solution, which is determined as an enzyme solution with high cellulase activity in the process (F) and the process (G) is an enzyme solution with higher cellulase activity.
[5] The method for assaying cellulase activity according to any one of [1] to [4], may further include:
   a process (M) of stopping the enzyme reaction by adding a reaction terminator to the substrate solutions and dispersing the reaction terminator therein, after the process of performing the enzyme reaction and before the process of measuring the absorbance after the enzyme reaction.
[6] In the method for assaying cellulase activity according to [5],
   the reaction terminator may be a strong alkaline solution at 0.5 mol/L to 2.5 mol/L.
[7] In the method for assaying cellulase activity according to any one of [1] to [6],
   the absorbance of each of the substrate solutions before and after the enzyme reaction may be measured at any one wavelength of 500 nm to 700 nm.
[8] In the method for assaying cellulase activity according to [7],
   the absorbance of each of the substrate solutions before the enzyme reaction which was measured in the process of measuring the absorbance of each of the substrate solutions may be 0.1 to 1.5.
[9] In the method for assaying cellulase activity according to any one of [1] to [8],
   the concentration of cellulose in the process of measuring the absorbance of each of the substrate solutions may be any one of 0.1 mass % to 30 mass % with respect to the substrate solutions.
[10] In the method for assaying cellulase activity according to any one of [1] to [9],
   the enzyme reaction may be performed by setting the reaction temperature to 30° C. to 60° C. and setting the reaction time to 15 minutes to 6 hours.
[11] In the method for assaying cellulase activity according to any one of [I] to [10],
   each of the substrate solutions may be fixed by a support body.
[12] In the method for assaying cellulase activity according to [11],
   the support body may be at least one selected from agar, gelatin, gellan gum, and glucomannan.
[13] In the method for assaying cellulase activity according to any one of [1] to [12],
   the cellulose may be phosphoric acid-swelling cellulose.
[14] In the method for assaying cellulase activity according to any one of [1] to [13],
   a reaction container of the enzyme reaction may be a microplate.
[15]A method for screening an enzyme solution, includes:
   comparing activities of enzyme solutions with each other using the method for assaying cellulase activity according to any one of [1] to [14].
[16]A method for screening cellulase-producing microorganisms, includes:
   comparing activities of microorganism culture solutions with each other using the method for assaying cellulase activity according to any one of [1] to [14].
[17] In the method for screening cellulase-producing microorganisms according to [16],
   the cellulase-producing microorganisms selected through the method for screening cellulase-producing microorganisms may be the genus Acremonium.
[18] In the method for screening cellulase-producing microorganisms according to [17],
   the microorganisms belonging to the genus Acremonium may be Acremonium cellulolyticus SD1745 NITE BP-01992 strains.
[19]Acremonium cellulolyticus SD1745 NITE BP-01992 strains which have cellulase activity.

According to the present invention, it is possible to simply and accurately assay cellulase activity. In addition, it is possible to obtain high-performance cellulase-producing bacteria through screening using the assay.

### BRIEF DESCRIPTION OF THE DRAWINGS

FIG. 1 is a flowchart describing a first embodiment of a method for assaying cellulase activity of the present invention.

FIG. 2 is a flowchart describing a second embodiment of the method for assaying cellulase activity of the present invention.

FIG. 3 is a flowchart describing a third embodiment of the method for assaying cellulase activity of the present invention.

FIG. 4 is a flowchart describing a fourth embodiment of the method for assaying cellulase activity of the present invention.

FIG. 5 is a flowchart describing a fifth embodiment of the method for assaying cellulase activity of the present invention.

FIG. 6 is a graph showing a relationship between an absorbance decrease value and a dilution ratio of a reference enzyme solution to a preparation enzyme solution in Example 1.

FIG. 7 is a graph showing a relationship between a hollow diameter and FPU activity in Example 2.

FIG. 8 is a graph showing a relationship between the absorbance decrease value and the FPU activity in Example 2.

FIG. 9 is a graph showing a relationship between the absorbance decrease value and a dilution ratio of a reference enzyme solution, S1745 strains, and S1789 strains in Example 3.

### DETAILED DESCRIPTION OF THE INVENTION

### Method for Assaying Cellulase Activity

### First Embodiment

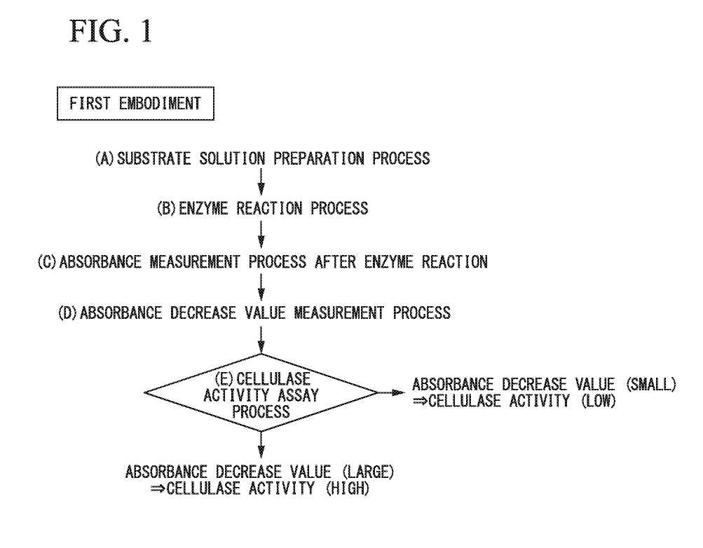

A method for assaying cellulase activity of a first embodiment of the present invention will be described with reference to FIG. 1. First, two or more substrate solutions, which have an identical absorbance measured at an identical wavelength and in which cellulose is dispersed at an identical concentration, are prepared, and the absorbance of each of the substrate solutions is measured (process A). Next, different kinds of enzyme solutions are respectively added to the substrate solutions, and an enzyme reaction is performed under the same conditions (process B). Thereafter, the absorbance of each of the substrate solutions after the enzyme reaction is measured (process C), and the absorbance decrease values of the substrate solutions before and after the enzyme reaction are calculated (process D). Lastly, cellulase activities of the enzyme solutions are assayed based on the absorbance decrease values (process E). In the assay of the process E, it is determined that the enzyme solutions have higher cellulase activity as the absorbance decrease values become larger.

In the present embodiment, the assay of cellulase activities is performed by unifying the wavelength at which the absorbance is measured, the absorbance before the enzyme reaction, the concentration of cellulose in the process A, and the conditions of the enzyme reaction, for each sample. However, there may be slight deviation to the extent that the assay is not affected.

In the present specification, the “substrate solution” refers to a solution in which cellulose is dispersed in a buffer solution or water using the cellulose as a substrate.

Cellulose is not limited as long as cellulose is evenly dispersed in a substrate solution and has absorbance which is decreased through an enzyme reaction. For example, it is possible to use Avicel of powdery crystalline cellulose, cellulose nanofibers, powdered cellulose-based biomass, and the like.

In a case of using a buffer solution as a solution in which cellulose is dispersed, it is possible to use, for example, an acetate buffer solution, a phosphate buffer solution, a citrate buffer solution, and a Tris-hydrochloric acid buffer solution, and it is preferable to use an acetate buffer solution.

It is preferable that cellulose be swollen with a phosphoric acid aqueous solution or an alkali aqueous solution. That is, it is preferable that cellulose be phosphoric acid-swelling cellulose or alkali-swelling cellulose. The phosphoric acid-swelling cellulose has favorable dispersibility of cellulose and rarely causes precipitation, and it is possible to more accurately measure the absorbance, which is more preferable.

The concentration of cellulose with respect to a substrate solution is preferably 0.1 mass % to 30 mass %, more preferably 0.1 mass % to 20 mass %, and still more preferably 0.1 mass % to 10 mass %.

It is preferable to fix a substrate solution, in which cellulose is dispersed, using a support body. If the substrate solution is fixed using the support body it is possible to prevent precipitation of cellulose and for an enzyme reaction to favorably progress. Examples of the support body with such an effect include agar, gelatin, gellan gum, and glucomannan.

The ratio of the support body with respect to the substrate solution is preferably 1 mass % to 15 mass %, more preferably 1 mass % to 10 mass %, and still more preferably 1 mass % to 5 mass %.

In the present specification, the “absorbance” is measured using a general absorption spectrometer. For example, it is possible to measure the absorbance using a microplate reader (Infinite M200, manufactured by Tecan Trading AG). The absorbance is preferably measured at 500 nm to 700 nm, more preferably 500 nm to 600 nm, and still more preferably 500 nm to 550 nm. If the wavelength is within the above-described range, the absorbance sensitivity of a soluble component becomes an appropriate value, and therefore, it is possible to accurately measure the absorbance. When measuring the absorbance, it is possible to use a solution or the like, which is obtained by adding water at the same volume as that of an enzyme solution, to a substrate solution, as a blank.

The absorbance of a substrate solution before an enzyme reaction is, from the viewpoint of accuracy of measurement, preferably 0.1 to 1.5, more preferably 0.2 to 1.0, and still more preferably 0.4 to 0.8. It is preferable that the absorbance of a substrate solution before an enzyme reaction be previously measured before adding an enzyme solution thereto.

In the present specification, the “enzyme solution” is not particularly limited as long as it is a solution, such as a microorganism culture solution, an enzyme formulation aqueous solution, an enzyme treatment process solution, or an enzyme purification process solution, which contains cellulase. From the viewpoint of analysis accuracy, it is preferable to use a supernatant from which impurities or sediments are removed. The amount of enzyme solution to be added is, with respect to 100 μL of a substrate solution in which cellulose is dispersed, preferably 1 μL to 15 μL, more preferably 1 μL to 10 μL, and still more preferably 1 μL to 5 μL.

The concentration of the enzyme solution is not particularly limited. In a case where it is difficult to measure the absorbance decrease value, the enzyme solution may be diluted or the concentration of cellulose may be adjusted.

In addition, assay may be performed by arranging the concentrations of component enzymes of a part of a sample. With the arrangement of the concentrations of component enzymes of a part of a sample, it is possible to more accurately assay activities of other component enzymes. In this case, it is preferable to arrange the concentrations of the component enzymes of the part of the sample to, for example, 0 U/L to 20 U/L, more preferably 1 U/L to 15 U/L, and still more preferably 5 U/L to 10 U/L. Examples of the component enzymes include β-glucosidase (BGL), cellobiohydrolase (CBH), and endoglucanase (EG).

When adding an enzyme solution to a substrate in which cellulose is dispersed, the pH may be maintained using a buffer solution. The buffer solution is not particularly limited as long as there is no component inhibiting enzyme activity and the buffer solution has a function capable of maintaining the pH to be an optimum value. A specific example of the buffer solution is as the above.

In the present specification, the “enzyme reaction” is performed in accordance with a well-known method. For example, the enzyme reaction can be performed using a method in which a reaction container is put into a thermostatic chamber, an oven, or the like of which the temperature is set to a reaction temperature, and the reaction container is shaken.

The temperature of the enzyme reaction is preferably 30° C. to 60° C., more preferably 40° C. to 60° C., and still more preferably 40° C. to 50° C.

The duration for the enzyme reaction is preferably 15 minutes to 6 hours, more preferably 30 minutes to 2 hours, and still more preferably 50 minutes to 70 minutes.

As the reaction container in the enzyme reaction, it is possible to use a test tube, a flask, a beaker, a Petri dish, and a microplate. It is possible to perform enzyme reactions many times at the same time and to measure the absorbances thereof in the same container, and therefore, it is preferable to use a microplate.

In the present specification, the “absorbance decrease value” indicates an absorbance decrease value after an enzyme reaction of a substrate solution with respect to before the enzyme reaction. For example, it is possible to calculate the absorbance decrease value by subtracting the absorbance before the enzyme reaction from the absorbance after the enzyme reaction.

In the present embodiment, it is possible to determine that the cellulase activity becomes higher as the enzyme solution absorbance decrease value becomes higher. This is supported by the fact that a proportional relationship is generally recognized between the absorbance decrease value and filter paper decomposition activity (FPU activity).

In contrast, as an example of an easy assaying method in the related art, there is an assaying method using a hollow diameter. This assaying method is useful in primary screening except for a sample having low FPU activity. However, there is no proportional relationship found between the size of the hollow diameter and the FPU activity.

Accordingly, it can be said that the assaying method of the present embodiment is advantageous in that it is possible to accurately assay FPU activity through a simple operation.

In the assay of cellulase activity of the present embodiment, it is possible to perform a comparison in activities of samples, of which a component enzyme is unknown, without preparing a reference sample. However, a reference sample may be prepared to be set as a standard of the activity.

### Second Embodiment

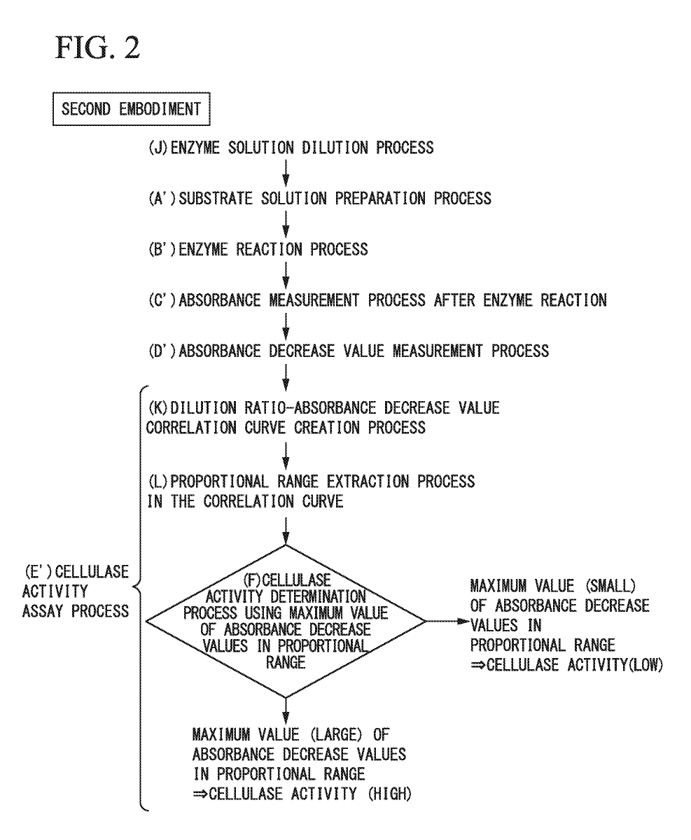

A method for assaying cellulase activity of a second embodiment of the present invention will be described with reference to FIG. 2. First, six or more diluents, which have different dilution ratios for each identical enzyme solution with respect to different kinds of enzyme solutions to be assayed, are prepared (process J). Next, six or more substrate solutions, which have an identical absorbance measured at an identical wavelength and in which cellulose is dispersed at an identical concentration, are prepared, and the absorbance of each of the substrate solutions is measured (process A′). Processes B′ to D′ performed next are the same as the processes B to D of the first embodiment. Furthermore, the cellulase activities of the enzyme solutions are assayed (process E′).

In the assay performed in the process E′, correlation curves between the dilution ratios of the enzyme solutions and the absorbance decrease values are created (process K).

A range in which the dilution ratios and the absorbance decrease values are in a proportional relationship is extracted in the correlation curves (process L). It is determined that an enzyme solution has higher cellulase activity as the maximum value in the range of the absorbance decrease values become larger, based on the correlation curves in the range (process F).

In the present specification, “diluents of an enzyme” are solutions obtained by diluting enzyme solutions, of which the cellulase activities need to be assayed, so as to have different concentrations from each other, and six or more solutions are prepared. The interval of the dilution ratios between the diluents is preferably set to 10% to 30%, and it is possible to set the interval to, for example, 0%, 20%, 40%, 60%, 80%, and 100%. However, the diluent at 0% represents a solution to which no enzyme solution is added.

The dilution ratio can be calculated as a percentage of the capacity of a solution before dilution with respect to the capacity of the solution after the dilution.

In the present specification, the “correlation curve” is a curve indicating a correlation relationship between a dilution ratio of an enzyme solution and an absorbance decrease value. It is preferable to set the horizontal axis as a dilution ratio and the longitudinal axis as an absorbance decrease value from the viewpoint of visibility.

It is also possible to create a correlation curve of a sample, of which a component enzyme is unknown, without creating a reference correlation curve and to use the correlation curve of the sample in assaying of cellulase activity. However, a reference correlation curve may be prepared to be set as a standard of the activity.

In addition, the correlation curve may be created by arranging activities of component enzymes of a part of a sample.

In the present specification, the “range in a proportional relationship” indicates a region which can be visually determined as a straight line in the correlation curve.

When extracting a range which becomes a proportional relationship, in a case where it is difficult to extract the range thereof since the inclination of a graph is too large or too small, the graph may be recreated by changing the dilution ratio.

It is possible to determine that the cellulase activity becomes higher as the maximum value of the absorbance decrease value increases in a range in which the dilution ratio and the absorbance decrease value are in a proportional relationship. To be more specific, it is possible to determine that the β-glucosidase (BGL) activity is high.

If the BGL activity is high, decomposition of cellobiose, which is a product of cellobiohydrolase (CBH), into glucose is accelerated and inhibition of the product of CBH tends to be released. If the inhibition of the product of CHB is released, decomposition of cellooligosaccharide, which is a product of endoglucanase (EG), into cellobiose is accelerated and inhibition of the product of EG tends to be released. Accordingly, cellulose decomposition properties of the whole cellulase are improved.

### Third Embodiment

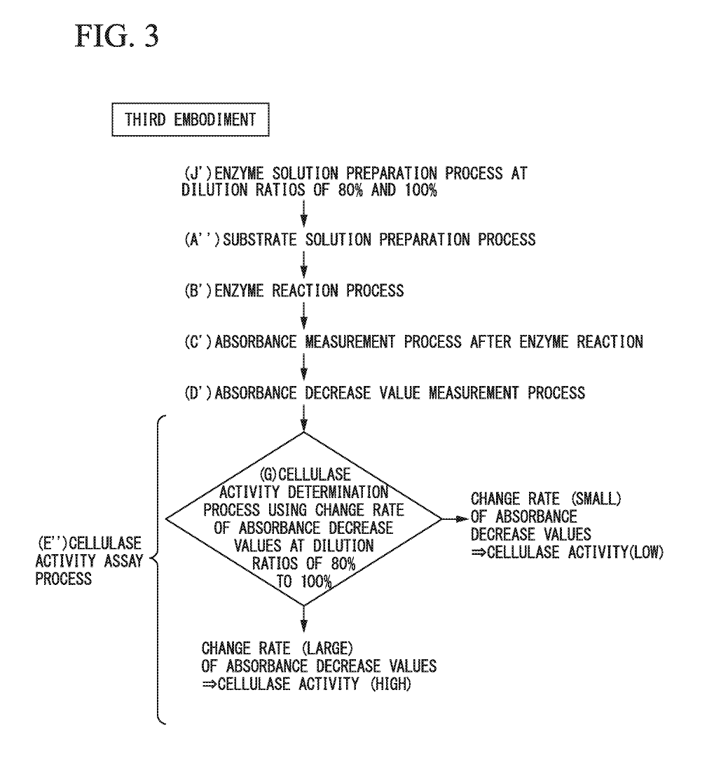

A method for assaying cellulase activity of a third embodiment of the present invention will be described with reference to FIG. 3. First, diluents, which are at dilution ratios of 80% and 100% for each identical enzyme solution with respect to different kinds of enzyme solutions to be assayed, are prepared (process J′). Next, two or more substrate solutions, which have an identical absorbance measured at an identical wavelength and in which cellulose is dispersed at an identical concentration, are prepared, and the absorbance of each of the substrate solutions is measured (process A″). Processes B″ to D″ performed next are the same as the processes B to D of the first embodiment and processes B′ to D′ of the second embodiment.

In the assay performed in the process E″, it is determined that the cellulase activity becomes higher as the change rate of the absorbance decrease values at the dilution ratios of 80% to 100% of the enzyme solutions (process G) becomes larger. To be more specific, it is possible to determine that the CBH activity is high.

If the CBH is high, decomposition of cellooligosaccharide, which is a product of EG, into cellobiose is accelerated and inhibition of the product of EG tends to be released. Accordingly, cellulose decomposition properties of the whole cellulase are improved.

### Fourth Embodiment

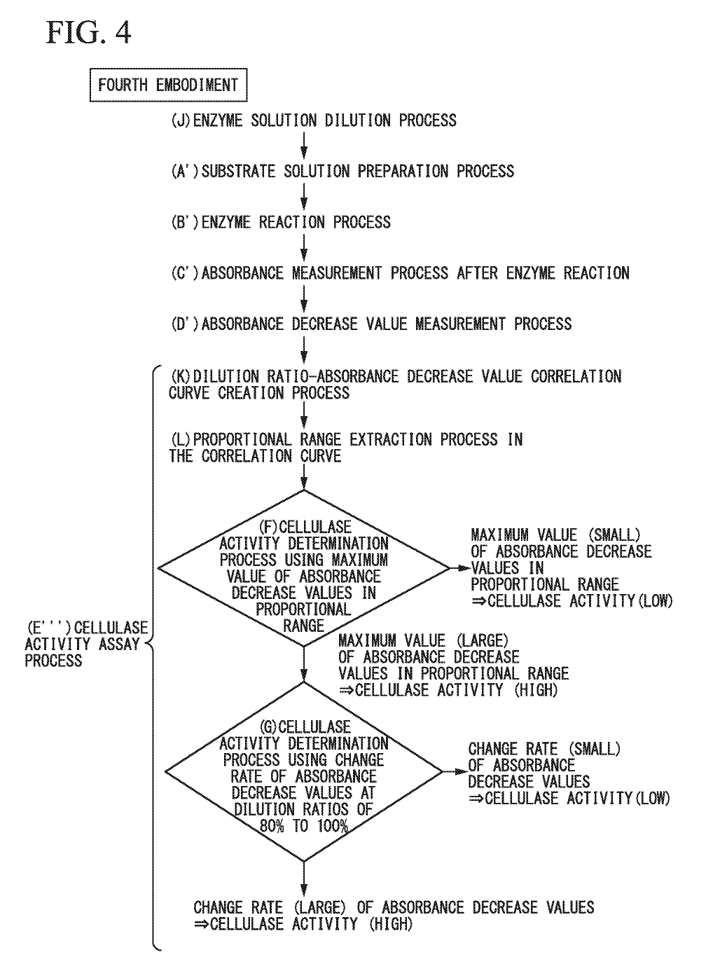

A method for assaying cellulase activity of a fourth embodiment of the present invention will be described with reference to FIG. 4. In the present embodiment, it is determined that enzyme solutions, which are determined to be an enzyme solution with high cellulase activity in the process F of the second embodiment and to be an enzyme solution with high cellulase activity in the process G of the third embodiment, are enzyme solutions with the highest cellulase activity, including the configurations of the second embodiment and the third embodiment. The enzyme solutions which are determined to have high cellulase activity in the present embodiment also have a correlation with saccharification performance.

In a case where the enzyme solutions which are determined to have high cellulase activity are different in the second embodiment and the third embodiment, it is determined that an enzyme solution having a large absorbance decrease value in the dilution ratio of 100% is an enzyme solution having high cellulase activity.

### Fifth Embodiment

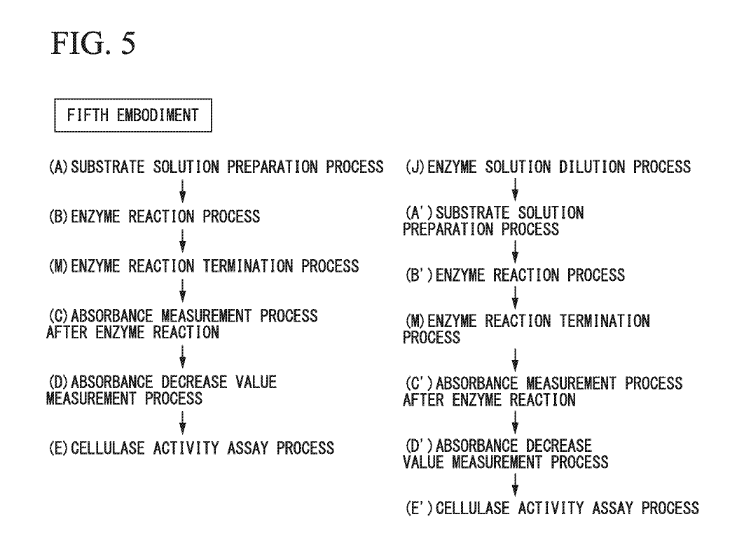

A method for assaying cellulase activity of a fifth embodiment of the present invention will be described with reference to FIG. 5. The enzyme reaction is stopped by adding a reaction terminator to the substrate solutions and dispersing the reaction terminator therein, after the process B or B′ which is a process of performing the enzyme reaction and before the process C or C′ which is a process of measuring the absorbance after the enzyme reaction (process M).

The enzyme reaction is preferably stopped by dispersing a reaction terminator on a substrate. The work such as measurement of the absorbance thereafter is efficiently performed by stopping the reaction.

Assay can be performed similarly to the first to fourth embodiments described above.

As the reaction terminator, it is possible to use, for example, a strong alkaline solution, and specifically NaOH and KOH.

The concentration of the reaction terminator is preferably 0.5 mol/L to 2.5 mol/L, more preferably 0.75 mol/L to 1.25 mol/L, and still more preferably 0.75 mol/L to 1.0 mol/L. Within the range, it is possible to reliably stop the reaction and the absorbance decrease value is not affected, which is preferable.

The amount of reaction terminator is, with respect to 100 μL of a substrate solution in which cellulose is dispersed, preferably 1 μL to 15 μL, more preferably 1 μL to 10 μL, and still more preferably 1 μL to 5 μL.

### Screening

### First Embodiment

It is possible to screen a large amount of sample at a time using the first embodiment or the second to fifth embodiments of the method for assaying cellulase activity. Specifically, it is possible to compare activities between an enzyme solution and a microorganism culture solution.

### Selection of Microorganisms

### First Embodiment

It is possible to select high-performance cellulase-producing bacteria using the screening method. Specifically, it is possible to use the following procedures.

### (1) Culture of Microorganisms as New Strains, and Mutation Treatment

(2) Primary Screening through Measurement of Hollow Diameter
(3) Secondary Screening through Measurement of Absorbance Decrease Value

Each of the above-described (1), (2), and (3) may be repeated, or a set of (1) to (3) may be repeated. In addition, (2) may be skipped depending on the situation. Each of the procedures will be described in detail below.

### (1) Culture of Microorganisms as New Strains, and Mutation Treatment

Microorganisms as new strains are not particularly limited as long as the microorganisms exhibit cellulase activity. For example, it is possible to use microorganisms belonging to the genus Acremonium, the genus Trichoderma, the genus Aspergillus, the genus Penicillium, the genus Irpex, the genus Bacillus, the genus Humicola, or the genus Ramicola. The genus Acremonium and the genus Trichoderma are preferable from the viewpoint of a high cellulase-producing ability, and Acremonium cellulolyticus S-1745 strains which are obtained as mutant strains of Acremonium cellulolyticus TN strains, or mutant strains thereof are more preferable.

The method for culturing microorganisms as new strains is not particularly limited as long as it is possible to culture selected microorganisms. As medium components used in culturing, it is possible to use well-known components. As the culture temperature, the pH, and the culture time, well-known conditions can be set.

For mutation treatment, it is possible to use an arbitrary method which is generally known. Examples of the method include irradiation with ultraviolet rays or activation of a mutation treatment agent such as N-methyl-N′-nitro-N-nitrosoguanidine. Alternately, the method may be performed through a gene recombination operation. As the operation procedure, it is possible to refer to “Molecular Biology Experimental Protocol I, II, and III” published by Maruzen Co., Ltd., (1997) or the like.

### (2) Primary Screening Through Measurement of Hollow Diameter

The primary screening may be performed on a culture solution obtained in “(1) Culture of Microorganisms as New Strains, and Mutation Treatment” using a well-known hollow diameter measurement method. As a hollow formation medium, it is possible to use, for example, a medium in which Avicel of crystalline cellulose, acid-swelling cellulose, alkali-swelling cellulose, cellulose nanofibers, Cellulose Azure (manufactured by SIGMA Corporation) which is pigment-bonding cellulose, obtained by bonding a blue pigment Remazol Brillianbt Blue R to cellulose, or the like is dispersed as a substrate.

In the screening, a culture solution of which the formed hollow diameter is larger than that formed using new strains may be selected.

### (3) Secondary Screening Through Measurement of Absorbance Decrease Value

Assay of cellulase activity was performed using the culture solution of microorganisms which had been selected in “(2) Primary Screening through Measurement of Hollow Diameter” through the procedures of the first embodiment or the second to fifth embodiments of the <<Method for Assaying Cellulase Activity>>. It is preferable to select microorganisms with high saccharification performance by performing assay of cellulase activity through the second to fifth embodiments.

### Acremonium Cellulolyticus S-1745 Strains

In the present invention, Acremonium cellulolyticus S-1745 strains which had cellulase activity with high saccharification performance were selected using the method for selecting microorganisms. Culturing was performed for 14 days using a potato dextrose flat-plate medium (dextrose (Difco Laboratories): 24 g/L, agar: 15 g/L, and pH: 5.6±0.2), and the form thereof was observed.

The microbiological properties of the Acremonium cellulolyticus S-1745 strains are as follows.

### (Morphological Qualitative Properties)

(1) Vegetative mycelia (mycelia in a state of vigorously proliferating under the conditions of rich nutrients in a favorable growing environment) of Acremonium cellulolyticus S-1745 strains have a partition wall, are colorless, and the surfaces of which are in smooth shapes.
(2) The colony of the Acremonium cellulolyticus S-1745 strains had a diameter of about 40 mm to 60 mm, had a yellowish white color to a white color, was flat, and was in velvet shape exhibiting a white hill in the central portion. In addition, infiltration into the lower portion of a medium was also recognized.

### (Reproduction Mode)

### (1) Proliferation Through Binary Division was Performed.

### (Physiological Biochemical Characteristics)

(1) Growth temperature range: 15° C. to 43° C. (optimum temperature is about 30° C.)
(2) Growth pH range: pH 3.5 to 6.0 (optimum pH is about 4.0)

From the above points, Acremonium cellulolyticus SD1745 strains were identified as ascomycetes of the genus Acremonium through an observation of the form thereof or the like.

The base sequence of an 18S rDNA gene of the Acremonium cellulolyticus S-1745 strains is shown in SEQ ID No: 1 of a sequence table.

The 18S rDNA gene of the Acremonium cellulolyticus S-1745 strains was compared with an 18S rDNA gene of closely related Acremonium cellulolyticus Y-94 strains (accession No. AB474749). The result was that the homology rate was 99.3%. However, it was determined that the Acremonium cellulolyticus SD1745 strains were new ascomycetes strains which were not the same kinds of Acremonium cellulolyticus Y-94 strains.

The Acremonium cellulolyticus SD1745 strains have been internationally deposited in the Patent Organism Depository of the National Institute of Technology and Evaluation (2-5-8 Kazusakamatari, Kisarazu-shi, Chiba-ken, Japan) since Jan. 14, 2015, with a deposition number of NITE BP-01992 under the Budapest Treaty.

### EXAMPLES

Hereinafter, the present invention will be described using Examples, but is not limited to the following Examples.

### Example 1

### Assay Using Correlation Curve

### 1-1. Preparation of Reference Enzyme Solution

Acremonium cellulolyticus TN strains (FERM P-18508; hereinafter, denoted as TN strains) were aerobically cultured for 6 days at 30° C. using a 018 mm test tube into which 5 mL of a liquid medium was put. After the culturing, the bacterial bodies were removed through centrifugal separation, and a supernatant was obtained and was set as a reference enzyme solution. The composition of the liquid medium is as follows.

### (Liquid Medium)

### Avicel: 50 g/L

KH₂PO₄: 24 g/L
Ammonium sulfate: 5 g/L
Potassium tartrate ½ H₂O: 4.7 g/L

### Urea: 4 g/L

### Tween 80: 1 g/L

MgSO₄.7H₂O: 1.2 g/L
ZnSO₄.7H₂O: 10 mg/L
MnSO₄.5H₂O: 10 mg/L
CuSO₄.5H₂O: 10 mg/L

### 1-2. Preparation of Preparation Enzyme Solution

Meicelase (cellulase base powder manufactured by Meiji Co., Ltd.) was dissolved in a 50 mM acetate buffer solution (pH 5) such that the concentration of the Meicelase became 15.6 mg/mL. This solution was mixed with the reference enzyme solution at a volume ratio of 1:1 to prepare an enzyme solution. The obtained enzyme solution was set as a preparation enzyme solution 1.

Next, a preparation enzyme solution was prepared which was obtained by dissolving BGL base powder (β-glucosidase, product No. 306-50981, manufactured by Wako Pure Chemical Industries, Ltd.) in a reference enzyme solution such that the concentration of the BGL base powder became 2 mg/mL. This enzyme solution was set as a preparation enzyme solution 2.

Next, a preparation enzyme solution was prepared which was obtained by dissolving BGL base powder in the preparation enzyme solution 1 such that the concentration of the BGL base powder became 2 mg/mL. This enzyme solution was set as a preparation enzyme solution 3.

### 1-3. Measurement of FPU Activity in Reference Enzyme Solution and Preparation Enzyme Solution.

The FPU activity is decomposition activity of filter paper due to cellulase, and the amount of enzyme generating reducing sugar, which corresponds to 1 μmol glucose, for 1 minute is defined as 1 unit (FPU: filter paper unit).

In measurement, Measurement of Cellulase Activities, Laboratory Analytical Procedure (LAP) of the National Renewable Energy Laboratory (NREL) in the United States was used. An enzyme reaction was performed by adding a sodium citrate buffer, and enzyme solutions which were prepared in 1-1 and 1-2, to a cap-attached test tube into which filter paper (Whatman No. 1) cut to 1 cm×6 cm was put. A dinitrosalicylic acid reagent was added to the solution after the reaction, and the mixture was heated at 100° C. and was colored. Thereafter, the mixture was cooled down using iced water, and the colored solution was diluted with pure water. The absorbance of the obtained solution at a wavelength of 540 nm was measured and the FPU activity was calculated using a calibration curve which had been created in advance.

The measurement results of the FPU activities in the reference enzyme solution and the preparation enzyme solutions 1 to 3 are shown in Table 1.

TABLE 1

| | Reference | Preparation | Preparation | Preparation |
|---|---|---|---|---|
| | enzyme | enzyme | enzyme | enzyme |
| | solution | solution 1 | solution 2 | solution 3 |
| FPU | 10 U/L | 10 U/L | 11 U/L | 11.5 U/L |
| activities | | | | |

### 1-4. Unification of FPU Activities of Reference Enzyme Solution and Preparation Enzyme Solution

In order to unify FPU activities of a reference enzyme solution and preparation enzyme solutions 1 to 3, the enzyme solutions were diluted with an acetic acid buffer solution such that the volume ratio of the preparation enzyme solution 2 became 1.1 times the reference enzyme solution and the volume ratio of the preparation enzyme solution 3 became 1.15 times.

1-5. Creation of Correlation Curve Using Reference Enzyme Solution and Preparation Enzyme Solution of which FPU Activities are Unified

### (1) Preparation of Phosphoric Acid-Swelling Cellulose

A solution was prepared by adding Avicel to ice-cooled phosphoric acid, and a suspension solution of phosphoric acid-swelling cellulose was prepared by adding the solution to 1900 L of ice-cooled water. The suspension solution was filtered under reduced pressure, pure water washing was performed four times on cellulose cake collected on the filtration surface, neutralizing and washing using a NaHCO₃ aqueous solution was performed twice, and then, pure water washing was further performed. After the washing, cellulose was collected and was subjected to homogenization treatment for 60 minutes at 12,000 rpm using Excel Auto Homogenizer ED-7 (500 mL SUS) manufactured by NISSEI Corporation, to be set as phosphoric acid-swelling cellulose. The solid content concentration of cellulose with respect to phosphoric acid-swelling cellulose was 33 mass %.

### (2) Preparation of Enzyme Solutions Having Different Dilution Ratios

Subsequently, a reference enzyme solution and a preparation enzyme solution of which FPU activities were unified were respectively diluted with a 50 mM acetate buffer solution to prepare diluents at dilution ratios of 0%, 5%, 10%, 20%, 30%, 40%, 50%, 60%, 80%, and 100% (undiluted). The dilution ratio is a percentage of the capacity before dilution to the capacity after dilution. A diluent at 0% is a solution with only a buffer solution.

### (3) Enzyme Reaction

Phosphoric acid-swelling cellulose (substrate solution) to which agar (support body) was added was used for reaction. The cellulose and the agar were prepared using acetate buffer solutions such that the contents of the cellulose and the agar respectively became 3.3 mass % and 1.2 mass % with respect to the substrate solution. The cellulose and the agar were heated and the agar was once dissolved in the acetate buffer solution. This was added to a 96-well microplate such that the amount thereof became 100 μL. 2 μL of each of the diluents of the above-described reference enzyme solution and the above-described preparation enzyme solution was added thereto. Then, the enzyme reaction was performed for 60 minutes at 50° C.

The absorbance (at a wavelength of 540 nm) of the substrate before the reaction was 0.57.

### (4) Measurement of Absorbance Decrease Value

After the enzyme reaction, 2 μL of 0.75 mol/L NaOH was added thereto to stop the reaction. The absorbance of the obtained solution (at a wavelength of 540 nm) was measured, and the absorbance decrease value during the reaction was calculated after subtracting the value of the absorbance of the obtained solution from the value thereof before the reaction. In the measurement of the absorbance, the absorbance of the sample which was set in the wells of the microplate was measured using a microplate reader (Infinite M200, manufactured by Tecan Trading AG). When measuring the absorbance, a solution, which was obtained by adding the same volume of water as that of the enzyme solution, to a substrate solution was used as a blank.

### (5) Creation of Correlation Curve

Correlation curves were created by setting the horizontal axis as a dilution ratio (%) of an enzyme and setting the longitudinal axis as an absorbance decrease value.

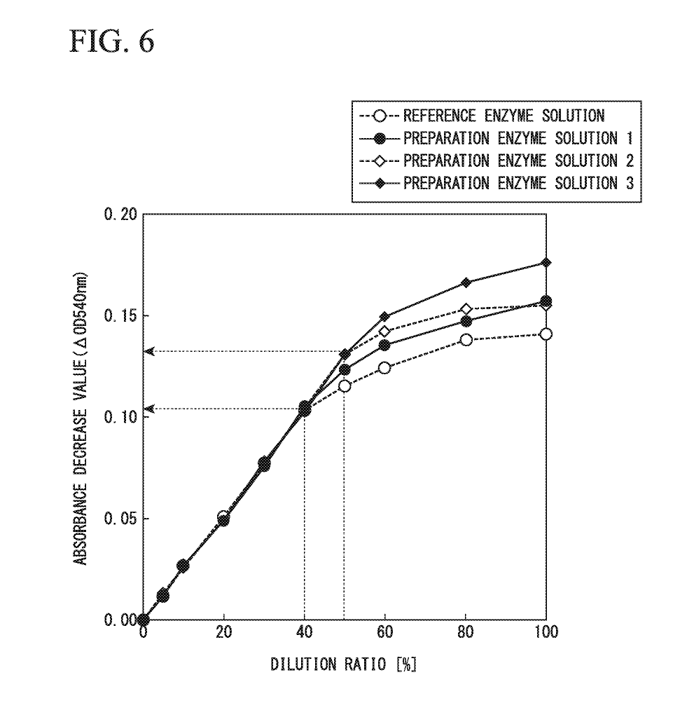

The correlation curves are shown in FIG. 6.

### 1-6. Assay

The maximum value of the absorbance decrease values in a range of a proportional relationship and the change rate of the absorbance decrease values at the dilution ratios of 80% to 100%, in each of the reference enzyme solution and the preparation enzyme solutions 1 to 3 are shown in Table 2. FIG. 6 was used for extracting the maximum value of the absorbance decrease values in a range of a proportional relationship.

In addition, the BGL activity, the CBH activity, the EG activity, and the saccharification rate of BM (ball mill) pulverization Avicel, in each of the enzymes were measured in order to check the accuracy of the assay. The results are shown in Table 2. The BGL activity, the CBH activity, the EG activity, and the saccharification rate of BM pulverization Avicel were calculated and measured through the methods shown below.

### (BGL Activity)

The BLG activity was calculated by quantitatively determining p-nitrophenol in which p-nitrophenyl-β-D-glucopyranoside (PNPG) was set as a substrate and which was isolated through an enzyme reaction. The absorbance was measured at a wavelength of 400 nm after adding an enzyme solution to a test tube, into which the p-nitrophenyl-β-D-glucopyranoside solution dissolved in an acetate buffer solution was put, for reaction, and adding a sodium carbonate aqueous solution and pure water. Subsequently, p-nitrophenol of the reaction solution was quantitatively determined based on the calibration curve, and the activity in which the amount of enzyme generating 1 μmol p-nitrophenol for one minute was set to one unit was calculated.

### (CBH Activity and EG Activity)

The CBH activity or the EG activity was calculated by quantitatively determining reducing sugar in which Avicel or carboxy cellulose was set as a substrate and which was isolated through an enzyme reaction. Specifically, the reaction was performed by putting an Avicel solution or a carboxy cellulose solution, which was dissolved in an acetate buffer solution, into a test tube, and adding an enzyme solution. Moreover, dinitrosalicylic acid reagent was added thereto and the mixture was heated at 100° C. and was colored. Thereafter, the absorbance at a wavelength of 540 nm was measured after ice-cooling the colored solution and diluting the cooled mixture with pure water. Subsequently, the activity was calculated after quantitatively determining the glucose of the reaction solution based on the calibration curve and setting the amount of enzyme which generated 1 μmol glucose for 1 minute as one unit.

### (Saccharification Rate of BM Pulverization Avicel)

The saccharification rate after reacting each of the enzyme solutions for 72 hours at 40° C. was measured in the presence of an acetate buffer solution using BM pulverization Avicel as a substrate. Specifically, the obtained saccharified liquid was analyzed through a well-known method using a glucose analyzer, a glucose measurement kit, or a well-known method such as high-speed liquid chromatography to quantitatively determine glucose, and the saccharification rate was calculated using the following numerical formula.

Saccharification rate=(concentration [%] of glucose of reaction solution)×0.9/(concentration [%] of cellulose before reaction)

TABLE 2

| | Ref- | | | |
|---|---|---|---|---|
| | erence | Preparation | Preparation | Preparation |
| | enzyme | enzyme | enzyme | enzyme |
| | solution | solution 1 | solution 2 | solution 3 |
| Maximum value of | 0.103 | 0.105 | 0.131 | 0.131 |
| absorbance decrease | | | | |
| values in range of | | | | |
| proportional | | | | |
| relationship | | | | |
| Change rate of | 0.003 | 0.010 | 0.002 | 0.010 |
| absorbance decrease | | | | |
| values at dilution | | | | |
| ratios of 80% to | | | | |
| 100% | | | | |
| BGL activity | 100 | 109 | 164 | 164 |
| (U/mL) | | | | |
| CBH activity | 3.0 | 4.6 | 3.0 | 4.6 |
| (U/mL) | | | | |
| EG activity (U/mL) | 130 | 131 | 130 | 131 |
| Saccharification | 76 | 79 | 83 | 86 |
| rate (%) of BM | | | | |
| pulverization Avicel | | | | |

### 1-7. Consideration of Assay Results

From Table 2, the maximum value of the absorbance decrease values in a range of a proportional relationship was large in preparation enzyme solutions 2 and 3. Accordingly, it was possible to determine that the BGL activity was high. The change rate of the absorbance decrease values at dilution ratios of 80% to 100% was large in preparation enzyme solutions 1 and 3. Accordingly, it was possible to determine that CBH was high. In the preparation enzyme solution 3, the maximum value of the absorbance decrease values in a range of a proportional relationship was high and the change rate of the absorbance decrease values at the dilution ratios of 80% to 100% was large. Accordingly, it was possible to determine that the saccharitfication performance is high.

In contrast, in the BGL activities separately measured, high values were shown in the preparation enzyme solutions 2 and 3. In the CBH activities, high values were shown in the preparation enzyme solutions 1 and 3. In the saccharification rate of BM pulverization Avicel, a high value was shown in preparation enzyme solution 3.

From the above, it was possible to confirm that there is a correlation between the assaying method of the present invention, and the activity of an actual component enzyme and the saccharification performance.

### Example 2

### Screening Through Measurement of Absorbance Decrease Value

### 2-1. Preparation of Mutant Strains

Culturing was performed for 24 hours at 30° C. using a 24 g/L a potato dextrose liquid medium (manufactured by Difco Laboratories) using TN strains as new strains. A mutation treatment strain solution was prepared by dispensing the obtained culture solution in a Petri dish and irradiating the solution with ultraviolet rays for 1 minute to 6 minutes.

### 2-2. Primary Screening Through Measurement of Hollow Diameter

The mutation treatment solution was diluted with physiological saline such that the concentration of bacterial bodies became 20 colony/ml to 200 colony/ml per agar medium, and culturing was performed using a hollow medium for 5 days at 30° C. The hollow in which cloudiness of cellulose became transparent was observed, and strains in which the hollow diameter was greater than or equal to 8 mm were selected.

A hollow medium having the following composition was used. The same phosphoric acid-swelling cellulose as that in Example 1 was used.

### (Hollow Medium)

Phosphoric acid-swelling cellulose: 5 g/L
KH₂PO₄: 2 g/L
Ammonium sulfate: 1.4 g/L
Potassium tartrate 1/2 H₂O: 4.7 g/L
Oxgall (manufactured by Difco Laboratories): 15 g/L
Peptone (manufactured by Difco Laboratories): 1 g/L.
CaCl₂.2H₂O: 0.4 g/L
MnSO₄.7H₂O: 0.3 g/L

### Tween 80: 0.2 g/L

### Urea: 0.4 g/L

ZnSO₄.7H₂O: 10 mg/L
MnSO₄.5H₂O: 10 mg/L
CuSO₄.5H₂O: 10 mg/L

### Agar: 20 g/L

### 2-3. Secondary Screening Through Measurement of Absorbance Decrease Value

The strains selected through the primary screening were cultured. The culturing was performed under the same conditions as those of the culturing performed in “2-2. Primary Screening through Measurement of Hollow Diameter”. After the culturing, the bacterial bodies were removed through centrifugal separation and supernatants were obtained. In each of the supernatants, the absorbance decrease value at a dilution ratio of 10% was measured. The dilution ratio is a percentage of the capacity before dilution to the capacity after dilution. The measurement of the absorbance decrease value was performed through the same method as that in Example 1. The reason why the dilution ratio of the solution for measuring the absorbance decrease value was set to 10% was because, if the dilution ratio was set to 10%, the FPU activity of new strains became 1 U/mL, and therefore, it was determined that it was easy to compare the activities from each other.

### 2-4. Assay

In Table 3, the absorbance decrease value of a new strain and the absorbance decrease values of strains of which the hollow diameter in the primary screening was greater than or equal to 8 mm are shown. In addition, the FPU activities were measured in order to check the accuracy of the assay. The results are shown in Table 3. The FPU activities were measured through the same method as that in Example 1.

TABLE 3

| | | Secondary | | FPU activity | |
|---|---|---|---|---|---|
| | Primary | screening | | measurement value | |
| | screening | | Proportion | | Proportion |
| | Hollow | Absorbance | with respect | | with respect |
| | Diameter | decrease | to TN strain | | to TN strain |
| Order | (mm) | value | (new strain) | U/mL | (new strain) |
| New strain | 7.0 | 0.026 | 100% | 10.0 | 100% |
| 1 | 8.0 | 0.038 | 148% | 14.3 | 143% |
| 3 | 8.0 | 0.037 | 142% | 13.4 | 134% |
| 4 | 8.5 | 0.034 | 130% | 12.9 | 129% |
| 5 | 8.5 | 0.032 | 124% | 12.8 | 128% |
| 6 | 9.0 | 0.032 | 123% | 12.5 | 125% |
| 7 | 11.0 | 0.034 | 130% | 12.3 | 123% |
| 8 | 8.0 | 0.031 | 120% | 12.3 | 123% |
| 9 | 8.0 | 0.032 | 122% | 12.3 | 123% |
| 10 | 11.0 | 0.028 | 108% | 12.0 | 120% |
| 11 | 10.0 | 0.029 | 110% | 12.0 | 120% |
| 12 | 8.5 | 0.032 | 123% | 11.5 | 115% |
| 13 | 8.0 | 0.029 | 110% | 11.0 | 110% |
| 14 | 8.5 | 0.028 | 106% | 11.0 | 110% |
| 15 | 8.0 | 0.030 | 114% | 10.5 | 105% |
| 16 | 10.0 | 0.026 | 100% | 9.5 | 95% |
| 17 | 10.0 | 0.026 | 100% | 9.5 | 95% |
| 18 | 3.5 | 0.022 | 85% | 9.0 | 90% |
| 19 | 8.0 | 0.021 | 80% | 8.5 | 85% |

### 2-5. Consideration of Assay Results

From Table 3, it is possible to determine that the cellulase activity was highest in the strain which showed an absorbance decrease value of 0.038.

In contrast, in the FPU activities separately measured, the highest value was shown in the strain which showed an absorbance decrease value of 0.038. Accordingly, it was possible to confirm that it is possible to accurately determine high values of the cellulase activities through the assaying method of the present invention.

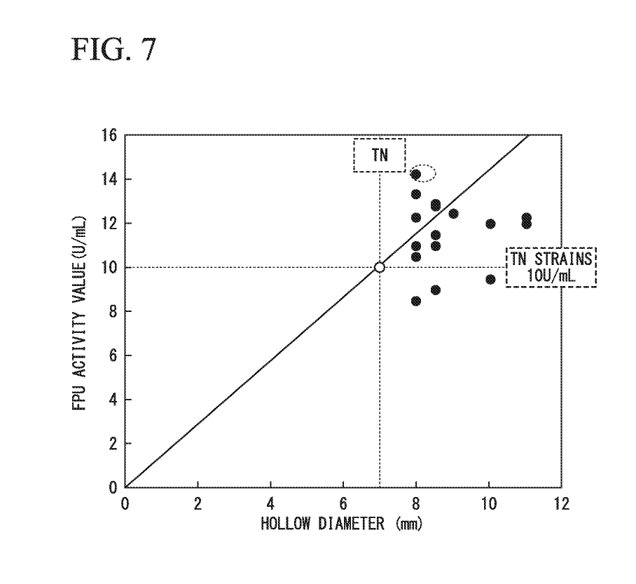

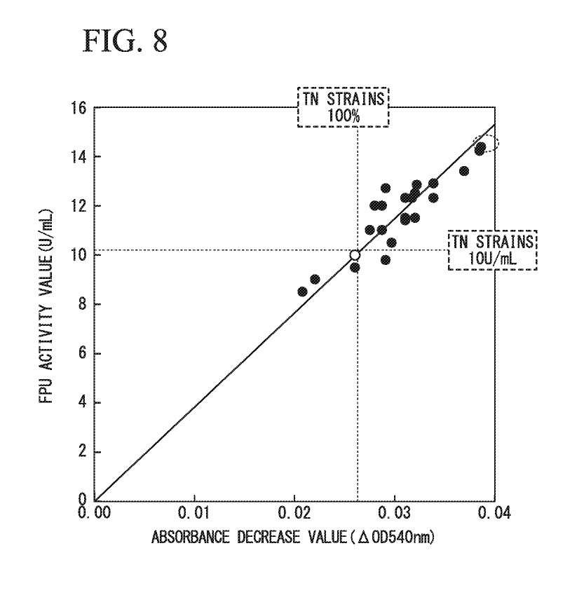

In addition, the relationship between the hollow diameter and the FPU activity value is shown in FIG. 7 and the relationship between the absorbance decrease value and the FPU activity is shown in FIG. 8.

From FIG. 7, there was not much correlation found between the size of the hollow diameter and the FPU activity value. In contrast, from FIG. 8, a general proportional relationship was found between the absorbance decrease value and the FPU activity. Accordingly, it was found that the assay using the hollow diameter is not suitable for accurately selecting a high strain of the FPU activity whereas the assay is suitable for the primary screening in which strains having low activity are excluded. In contrast, in the assay using the absorbance decrease value, it is possible to assay the FPU activity by checking the magnitude of the value, and therefore, the assay is suitable as the secondary screening.

### Example 3

### Selection of Microorganisms

### 3-1. Preparation of Mutant Strains, Primary Screening Through Measurement of Hollow Diameter, and Secondary Screening Through Measurement of Absorbance Decrease Value

Bacterial strains which showed the maximum value (0.038) of the absorbance decrease value in Example 2 were named as F17 strains. Preparation of mutant strains, primary screening through measurement of a hollow diameter, and secondary screening through measurement of an absorbance decrease value were performed on the F17 strains in the same manner as in Example 2.

As a result of the screening, an S1745 strain (absorbance decrease value: 0.059) with the highest absorbance decrease value and an S1789 strain (absorbance decrease value: 0.052) with the second highest absorbance decrease value were selected.

### 3-2. Creation of Correlation Curve

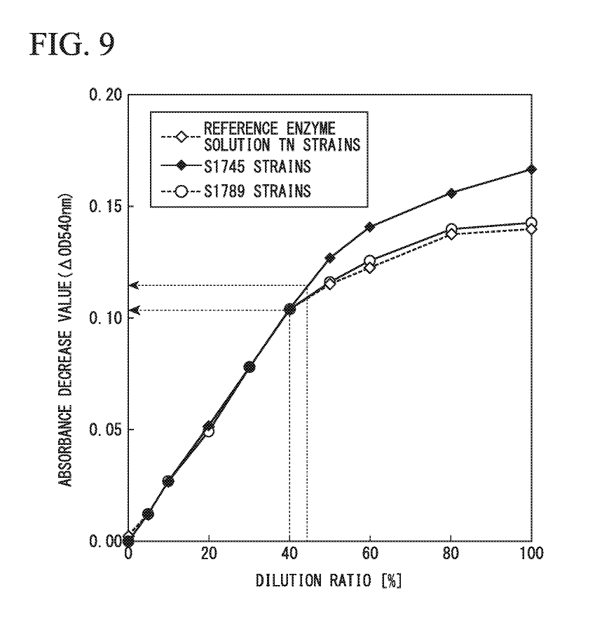

Regarding the TN strain as a new strain, and the S1745 strain and the S1789 strain which were selected through the screening, correlation curves were created in the same manner as in Example 1. The results are shown in FIG. 9.

### 3-3. Selection of Microorganisms

The maximum value of the absorbance decrease values in a range of a proportional relationship and the change rate of the absorbance decrease values at the dilution ratios of 80% to 100%, in the TN strain, as a new strain, the F1745 strain, and F1789 strain are shown in Table 4. When extracting the maximum value of the absorbance decrease values in a range of a proportional relationship, FIG. 9 was used.

In addition, the FPU activity, the BGL activity, the CBH activity, the EG activity, the saccharification rate of BM pulverization Avicel, and the saccharification rate of hydrothermal fine pulverization bagasse in each of the strains were measured in order to check the accuracy of the assay. The results are shown in Table 4. The FPU activity, the BGL activity, the CBH activity, the EG activity, and the saccharification rate of BM pulverization Avicel were measured through the same method as that in Example 1. The hydrothermal fine pulverization bagasse was measured through the same method as that of measuring the saccharification rate of BM pulverization Avicel except that the hydrothermal fine pulverization bagasse was used as a raw material.

TABLE 4

| | TN strain | S1745 strain | S1789 strain |
|---|---|---|---|
| Maximum value of absorbance | 0.103 | 0.127 | 0.104 |
| decrease values in range of | | | |
| proportional relationship | | | |
| Change rate of absorbance | 0.003 | 0.011 | 0.003 |
| decrease values at dilution | | | |
| ratios of 80% to 100% | | | |
| FPU activity (U/mL) | 10 | 22.1 | 19.8 |
| BGL activity (U/mL) | 100 | 320 | 220 |
| CBH activity (U/mL) | 3 | 15.5 | 6 |
| EG activity (U/mL) | 130 | 290 | 240 |
| Saccharification rate (%) | 76 | 85 | 77 |
| of BM pulverization Avicel | | | |
| Saccharification rate (%) | 70 | 85 | 74 |
| of hydrothermal fine | | | |
| pulverization bagasse | | | |

### 3-4 Consideration

From Table 4, the maximum value of the absorbance decrease values in a range of a proportional relationship in the S1745 strain is higher than that in the S1789 strain and the change rate of the absorbance decrease values at the dilution ratios of 80% to 100% in the S1745 strain is larger than that in the S1789 strain. Thus, according to the assaying method of the present invention, it is possible to select the S1745 strain as a high-performance cellulase-producing bacterium.

In contrast, the FPU activity, the BGL activity, the CBH activity, and the EG activity, which were separately measured, show high values in the S1745 strain. In addition, the saccharification rate of BM pulverization Avicel and the saccharification rate of hydrothermal fine pulverization bagasse show high values in the S1745 strain.

From the above, it was possible to confirm that it was possible to accurately select high-performance cellulase-producing bacteria through the assaying method of the present invention.

According to the present invention, it is possible to simply and accurately assay cellulase activity. In addition it is possible to obtain high-performance cellulase-producing bacteria through screening using the assay.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A method for assaying cellulase activity, comprising:
a process (A) of preparing two or more substrate solutions, which have an identical absorbance measured at an identical wavelength and in which cellulose is dispersed at an identical concentration, and measuring the absorbance of each of the substrate solutions;
a process (B) of respectively adding different kinds of enzyme solutions to the substrate solutions, and performing an enzyme reaction under the same conditions;
a process (C) of measuring the absorbance of each of the substrate solutions after the enzyme reaction;
a process (D) of calculating the absorbance decrease values of the substrate solutions before and after the enzyme reaction; and
a process (E) of assaying cellulase activities of the enzyme solutions based on the absorbance decrease values,
wherein, in the assay of the process (E), it is determined that the enzyme solutions have higher cellulase activity as the absorbance decrease values become larger.

2. A method for assaying cellulase activity, comprising:
a process (J) of preparing six or more diluents, which have different dilution ratios for each identical enzyme solution with respect to different kinds of enzyme solutions to be assayed;
a process (A′) of preparing six or more substrate solutions, which have an identical absorbance measured at an identical wavelength and in which cellulose is dispersed at an identical concentration, and measuring the absorbance of each of the substrate solutions;
a process (B′) of respectively adding the diluents to the substrate solutions and performing an enzyme reaction under the same conditions;
a process (C′) of measuring the absorbance of each of the substrate solutions after the enzyme reaction;
a process (D′) of calculating the absorbance decrease values of the substrate solutions before and after the enzyme reaction; and
a process (E′) of assaying cellulase activities of the enzyme solutions,
wherein the process (E′) includes
a process (K) of creating correlation curves between the dilution ratios of the enzyme solutions and the absorbance decrease values,
a process (L) of extracting a range in which the dilution ratios and the absorbance decrease values are in a proportional relationship in the correlation curves, and
a process (F) of assaying the cellulase activity of the enzyme solutions based on the correlation curves in the range and determining that an enzyme solution has higher cellulase activity as the maximum value in the range of the absorbance decrease values becomes larger.

3. A method for assaying cellulase activity, comprising:
a process (J′) of preparing diluents, which are at dilution ratios of 80% and 100% for each identical enzyme solution with respect to different kinds of enzyme solutions to be assayed;
a process (A″) of preparing two substrate solutions, which have an identical absorbance measured at an identical wavelength and in which cellulose is dispersed at an identical concentration, and measuring the absorbance of each of the substrate solutions;
a process (B′) of respectively adding the two diluents to the substrate solutions and performing an enzyme reaction under the same conditions;
a process (C′) of measuring the absorbance of each of the substrate solutions after the enzyme reaction;
a process (D′) of calculating the absorbance decrease values of the substrate solutions before and after the enzyme reaction; and
a process (E″) of assaying cellulase activities of the enzyme solutions,
wherein the process (E″) includes
a process (G) of determining that an enzyme solution has higher cellulase activity as the change rate of the absorbance decrease values at dilution ratios of 80% to 100% of the enzyme solutions becomes larger.

4. The method for assaying cellulase activity according to claim 3, further comprising:
a process (F) of assaying the cellulase activity of the enzyme solutions based on the correlation curves in the range and determining that an enzyme solution has higher cellulase activity as the maximum value in the range of the absorbance decrease values becomes larger,
wherein it is determined that an enzyme solution, which is determined as an enzyme solution with high cellulase activity in the process (F) and the process (G) is an enzyme solution with higher cellulase activity.

5. The method for assaying cellulase activity according claim 1, further comprising:
a process (M) of stopping the enzyme reaction by adding a reaction terminator to the substrate solutions and dispersing the reaction terminator therein, after the process of performing the enzyme reaction and before the process of measuring the absorbance after the enzyme reaction.

6. The method for assaying cellulase activity according to claim 5,
wherein the reaction terminator is a strong alkaline solution at 0.5 mol/L to 2.5 mol/L.

7. The method for assaying cellulase activity according to claim 1,
wherein the absorbance of each of the substrate solutions before and after the enzyme reaction is measured at any one wavelength of 500 nm to 700 nm.

8. The method for assaying cellulase activity according to claim 7,
wherein the absorbance of each of the substrate solutions before the enzyme reaction which was measured in the process of measuring the absorbance of each of the substrate solutions is 0.1 to 1.5.

9. The method for assaying cellulase activity according to claim 1,
wherein the concentration of cellulose in the process of measuring the absorbance of each of the substrate solutions is any one of 0.1 mass % to 30 mass % with respect to the substrate solutions.

10. The method for assaying cellulase activity according to claim 1,
wherein the enzyme reaction is performed by setting the reaction temperature to 30° C. to 60° C. and setting the reaction time to 15 minutes to 6 hours.

11. The method for assaying cellulase activity according to claim 1,
wherein each of the substrate solutions is fixed by a support body.

12. The method for assaying cellulase activity according to claim 11,
wherein the support body is at least one selected from agar, gelatin, gellan gum, and glucomannan.

13. The method for assaying cellulase activity according to claim 1,
wherein the cellulose is phosphoric acid-swelling cellulose.

14. The method for assaying cellulase activity according to claim 1,
wherein a reaction container of the enzyme reaction is a microplate.

15. A method for screening an enzyme solution, comprising:
comparing activities of enzyme solutions with each other using the method for assaying cellulase activity according to claim 1.

16. A method for screening cellulase-producing microorganisms, comprising:
comparing activities of microorganism culture solutions with each other using the method for assaying cellulase activity according to claim 1.

17. The method for screening cellulase-producing microorganisms according to claim 16,
wherein the cellulase-producing microorganisms selected through the method for screening cellulase-producing microorganisms are from the genus Acremonium.

18. The method for screening cellulase-producing microorganisms according to claim 17,
wherein the microorganisms belonging to the genus Acremonium are Acremonium cellulolyticus SD1745 NITE BP-01992 strains.

19. Acremonium cellulolyticus SD1745 NITE BP-01992 strains which have cellulase activity.


SEQUENCE LISTING (1 sequence)

SEQ ID NO: 1 - DNA, 603 bp, Acremonium cellulolyticus SD1745
ggaagtaaaagtcgtaacaaggtttccgtaggtgaacctgcggaaggatcattaccgagtgcgggccctcgcggcccaacctcccacccttgtctctatacacctgttgctttggcgggcccaccggggccacctggtcgccgggggacgcacgtctccgggcccgcgcccgccgaagcgctctgtgaaccctgatgaagatgggctgtctgagtactgtgaaaattgtcaaaactttcaacaatggatctcttggttccggcatcgatgaagaacgcagcgaaatgcgataagtaatgtgaattgcagaattccgtgaatcatcgaatctttgaacgcacattgcgccccctggcattccggggggcatgcctgtccgagcgtcatttctgccctcaagcacggcttgtgtgttgggtgtggtccccccggggacctgcccgaaaggcagcggcgacgtccgtctggtcctcgagcgtatggggctctgtcactcgctcgggaaggacctgcgggggttggtcaccaccatgttttaccacggttgacctcggatcaggtaggagttacccgctgaacttaagcatatcaataagcggagga